SEQUENCE LISTING (4 sequences)

SEQ ID NO: 1 - DNA, 20 bp, artificial
  forward primer
aaagaaagccaggaggggta

SEQ ID NO: 2 - DNA, 20 bp, artificial
  reverse primer
cggaggacaggtagaatgga

SEQ ID NO: 3 - DNA, 20 bp, artificial
  forward primer
atctcacagggagaggcaga

SEQ ID NO: 4 - DNA, 20 bp, artificial
  reverse primer
ccttgggataaccttgcaga

(19) United States
(12) Patent Application Publication
(10) Pub. No.: US 2008/0107630 A1
(43) Pub. Date: May 8, 2008

(54) HUMAN MATRIX METALLOPROTEINASE-8 GENE DELIVERY ENHANCES THE ONCOLYTIC ACTIVITY OF A REPLICATING ADENOVIRUS

(75) Inventors: John G. Hay, Baldwin Place, NY (US); Jin Cheng, Forest Hill, NY (US); Harald Sauthoff, White Plains, NY (US)

Correspondence Address: NIXON PEABODY LLP - PATENT GROUP, 1100 CLINTON SQUARE, ROCHESTER, NY, 14604, US

(73) Assignee: NEW YORK UNIVERSITY, New York, NY (US)

(21) Appl. No.: 11/697,394

(22) Filed: Apr. 6, 2007

Related U.S. Application Data
(60) Provisional application No. 60/790,629, filed on Apr. 10, 2006.

(51) Int. Cl. A61K 39/12; A61P 35/00; C12N 9/50

(52) U.S. Cl. 424/93.6; 435/219

(57) ABSTRACT

The present invention discloses a method of treating cancer in a subject. This involves co-administering a replicating virus and a matrix metalloproteinase to the subject under conditions effective to treat cancer. It also relates to a method of enhancing the delivery to and distribution within a tumor mass of therapeutic viruses. This involves co-administering a replicating virus and a matrix metalloproteinase to the tumor mass under conditions effective to enhance the delivery to and distribution within the tumor mass of therapeutic viruses. Another aspect relates to a cancer therapeutic. This involves a replicating virus and a matrix metalloproteinase.

This application claims the benefit of U.S. Provisional Patent Application Ser. No. 60/790,629, filed Apr. 10, 2006, which is hereby incorporated by reference in its entirety.

The subject matter of this application was made with support from the United States Government under The National Institutes of Health, Grant R01CA102053, and MO1RR-00096. The U.S. Government may retain certain rights in this invention.

### FIELD OF THE INVENTION

The present invention is directed to human matrix metalloproteinase gene delivery to enhance the oncolytic activity of a replicating virus.

### BACKGROUND OF THE INVENTION

Adenoviral vectors mediate gene transfer at a high efficacy compared to other vector systems, and they are currently the most frequently used vectors for cancer gene therapy. A non-replicating p53 expressing adenoviral vector (Peng, Z., “Current Status of Gendicine in China: Recombinant Human Ad-p53 Agent for Treatment of Cancers,” Hum Gene Ther 16:1016-1027 (2005)) and a replication selective virus (H101) have received regulatory approval in China (No authors listed, “The End of the Beginning: Oncolytic Virotherapy Achieves Clinical Proof-of-concept,” Mol Ther 13:237-238 (2006)). The success of recombinant adenoviruses in cancer therapy is, however, limited by inefficient delivery. The suboptimal transduction of cancer cells is compounded by poor distribution of the virus within the tumor mass (Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003)).

The use of replicating adenoviruses is a logical development, in that by repeated rounds of infection, release and re-infection of adjacent cells, the virus has the potential to spread from cell-to cell through the tumor mass, despite any initial problems with uniformity of delivery (Vile et al., “The Oncolytic Virotherapy Treatment Platform for Cancer: Unique Biological and Biosafety Points to Consider,” Cancer Gene Ther 9:1062-1067 (2002); Parato et al., “Recent Progress in the Battle Between Oncolytic Viruses and Tumours,” Nat Rev Cancer 5:965-976 (2005)). This is certainly the case in cell culture monolayers where a replicating virus can rapidly spread throughout a monolayer cell culture despite a low proportion of cells being initially infected. In contrast, xenograft tumors in immune incompetent mice are rarely eradicated despite the persistence of high levels of infectious virus within the tumors (Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003); Harrison et al., “Wild-type Adenovirus Decreases Tumor Xenograft Growth, but Despite Viral Persistence Complete Tumor Responses are Rarely Achieved—Deletion of the Viral E1b-19-kD gene Increases the Viral Oncolytic Effect,” Hum Gene Ther 12:1323-1332 (2001); Doronin et al., “Tumor-specific, Replication-competent Adenovirus Vectors Overexpressing the Adenovirus Death Protein,” J Virol 74:6147-6155 (2000)). Also in clinical studies, despite some evidence of replication and efficacy, overall results have been disappointing and the strategy has probably not reached its full potential (Chiocca et al., “A Phase I Open-label, Dose-escalation, Multi-institutional Trial of Injection with an E1B-attenuated Adenovirus, ONYX-015, into the Peritumoral Region of Recurrent Malignant Gliomas, in the Adjuvant Setting,” Mol Ther 10:958-966 (2004); Galanis et al., “Phase I-II Trial of ONYX-015 in Combination with MAP Chemotherapy in Patients with Advanced Sarcomas,” Gene Ther 12:437-445 (2005); Kim et al., “Clinical Research Results with dl1520 (Onyx-015), a Replication-selective Adenovirus for the Treatment of Cancer: What Have We Learned?,” Gene Ther 8:89-98 (2001)).

A previous study showed that after local injection of replicating-competent adenovirus into xenograft tumors, high levels of titratable virus could be recovered from a tumor as late as 100 days after initial viral injection. Tumors even persist at a time when infectious virus can be detected in the circulation several weeks after initial viral injection (Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003); Harrison et al., “Wild-type Adenovirus Decreases Tumor Xenograft Growth, but Despite Viral Persistence Complete Tumor Responses are Rarely Achieved—Deletion of the Viral E1b-19-kD gene Increases the Viral Oncolytic Effect,” Hum Gene Ther 12:1323-1332 (2001)). The viral persistence without tumor eradication suggested to applicants that viral spread is limited through tumor tissue, which is supported by the patchy and uneven intratumoral distribution of virus. Virus-infected cells can often be seen flanked by tumor necrosis and murine connective tissue. These data suggest that human adenoviral spread within tumor xenografts may be impaired by connective tissue (Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003)).

The present invention is directed to overcoming these and other deficiencies in the art.

### SUMMARY OF THE INVENTION

The present invention relates to a method of treating cancer in a subject. This involves co-administering a replicating virus and a matrix metalloproteinase to the subject under conditions effective to treat cancer.

The present invention also relates to a method of enhancing the delivery to and distribution within a tumor mass of therapeutic viruses. This involves co-administering a replicating virus and a matrix metalloproteinase to the tumor mass under conditions effective to enhance the delivery to and distribution within the tumor mass of therapeutic viruses.

Another aspect of the present invention relates to a cancer therapeutic. This involves a replicating virus and a matrix metalloproteinase.

The hypothesis that is addressed by the present invention is that matrix within the tumor hinders the free cell-to-cell spread of replicating adenoviral vectors. Matrix components within tumors include glycoproteins (tenascin, laminin, vitronectin, fibronectin), collagen types I-VI, (particularly collagen I and IV), and proteoglycans (Pupa et al., “New Insights into the Role of Extracellular Matrix During Tumor Onset and Progression,” J Cell Physiol 192:259-267 (2002), which is hereby incorporated by reference in its entirety).

Collagen I is a major component of tumor stroma, and interstitial collagen fibrils are resistant to degradation by most proteases. However, members of the fibrillar collagenase matrix metalloproteinase (MMP) family, in particular MMP-1, MMP-8, and MMP-13, can breakdown intact triple-helical collagen (Hasty et al., “The Collagen Substrate Specificity of Human Neutrophil Collagenase,” J Biol Chem 262:10048-10052 (1987); Owen et al., “Membrane-bound Matrix Metalloproteinase-8 on Activated Polymorphonuclear Cells is A Potent, Tissue Inhibitor of Metalloproteinase-resistant Collagenase and Serpinase,” J Immunol 172:7791-7803 (2004), which is hereby incorporated by reference in its entirety). MMP-8 is a Zn²⁺ metalloendopeptidase, predominantly expressed by neutrophils, but also by a few melanoma cell lines, chondrocytes, rheumatoid synovial fibroblasts, activated macrophages, smooth muscle cells, and endothelial cells (Herman et al., “Expression of Neutrophil Collagenase (matrix metalloproteinase-8) in Human Atheroma: A Novel Collagenolytic Pathway Suggested by Transcriptional Profiling,” Circulation 104:1899-1904 (2001), which is hereby incorporated by reference in its entirety). MMP-1 degrades type III collagen more efficiently then type I or type II collagen. MMP-8 cleaves types I, II, and III collagens with 20-fold selectivity for type I over type III, but MMP-8 does not degrade types IV or V collagen (Hasty et al., “The Collagen Substrate Specificity of Human Neutrophil Collagenase,” J Biol Chem 262:10048-10052 (1987), which is hereby incorporated by reference in its entirety). MMP-13, in turn, degrades type II collagen 6-fold more efficiently than type I or type III collagen.

Applicants have studied several matrix components to determine the role they may have on the efficacy of a replicating viral vector. It was found that collagen I can block adenoviral diffusion in in vitro experiments. Based on this finding, AdMMP8, a non-replicating adenoviral vector that expresses MMP-8, was constructed and shown to break down collagen in vitro. To evaluate the effects of MMP-8 expression from a non-replicating virus on the oncolytic activity of a replicating virus, AdMMP8 was administered in combination with wild type virus. Intratumoral injection of non-replicating AdMMP8 in combination with wild type virus in a murine xenograft tumor model, lead to reduced tumor cell growth and reduced expression of collagen within areas of virus induced necrosis compared to wild type virus treatment together with a non-replicating control.

The success of replicating adenoviruses for cancer therapy is limited by inefficient virus delivery. The initial suboptimal transduction of cancer cells is compounded by poor distribution of the virus within the tumor mass. Tumors consist of cancer cells, but also abundant amounts of stroma comprised of cells and matrix. This stromal matrix within the tumor, which includes collagen I, collagen IV, fibronectin, laminin, and proteoglycans, may hinder the free cell-to-cell spread of replicating adenoviral vectors. The present invention shows that collagen I, but not collagen IV, laminin, nor fibronectin, was able to block the passage of a non-replicating adenoviral vector through a membrane in in vitro cell culture experiments. Based on the effective collagen I-degrading activity of MMP-8, an adenovirus expressing the MMP-8 transgene was constructed. Established human lung cancer A549 xenografts were injected with a wild-type replicating adenovirus Adwt300 together with the non-replicating AdMMP8 virus. Co-infection of AdMMP8 with the wild type virus significantly reduced the growth of tumors compared with control tumors injected with the wild type virus in combination with a non-replicating control virus. AdMMP8 injection alone did not affect the growth of the lung A549 tumor xenografts. Histochemical analysis demonstrated reduced amounts of collagen within necrotic areas of MMP-8 injected tumors compared to controls. Therefore, these results demonstrate that intratumoral AdMMP8 and collagen disruption is a possible strategy for improving viral spread and improving the oncolytic activity of replicating adenovirus.

### BRIEF DESCRIPTION OF THE DRAWINGS

FIGS. 1A-F shows a virus diffusion assay utilizing BD Biocoat™ cell culture inserts and the in situ staining of cells for β-galactosidase. 293 cells were plated on the base of 24 well plates. After 24 h incubation, inserts containing Adβ-gal virus (MOI 5) diluted in 0.3 ml of MEM medium were placed onto the wells. The inserts had a 3 μm pore size and were pre-coated with collagen I, collagen IV, fibronectin, and laminin. The control insert had no coating. After 24 h infection, the ability of virus spread through the insert was analyzed. Collagen I markedly reduces the proportion of cells expressing β-gal (FIG. 1E) compared to control (FIG. 1A) and other matrix components (FIG. 1B-D), and this can be restored to control levels by pre-treatment with collagenase (FIG. 1F).

FIG. 2 shows a quantitative β-galactosidase assay. Collagen I significantly (p<0.0001) reduced β-gal expression compared to the other matrix components.

FIG. 3A shows a schematic of AdMMP8, a non-replicating adenovirus expressing the human MMP-8 full-length cDNA under the control of a CMV promoter. FIG. 3B shows A549 cell lines infected with AdMM8 express MMP-8 mRNA as detected by RT-PCR (lane 3). The expression of GAPDH is shown as a control. Control non-infected cell PCR is shown in lane 1, and control vector infected cells in lane 2. FIG. 3C shows medium from cultures of infected cells were separated on a 10% SDS-PAGE glycine gel for immunoblotting with a goat polyclonal anti-MMP-8 antibody. Evidence of MMP-8 protein expression is seen in the AdMMP8 infected group in lane 3. FIG. 3D shows supernatants from AdMMP8 infected cells display collagen degrading activity (lane 3) as depicted on a 10% SDS-PAGE glycine with 0.1% gelatin gel (zymogen gel).

FIG. 4 shows AdMMP8 conditioned media breaks down fibrillar collagen. Fibrillar collagen inserts conditioned by A549 cells infected with AdMMP8, but not Adcon infected cells, facilitate diffusion of an adenoviral reporter construct.

FIG. 5A shows a Kaplan-Meier cumulative survival plot and log rank testing. The data shows a significant increase in survival of animals treated with AdMMP8/Adwt300 compared to vehicle injected controls (p=0.004) and Adwt300/Adcon (p=0.008) injected animals based on the time to a three-fold increase in tumor volume. FIG. 5B shows a tumor growth curve. At day 26, tumors injected with Adwt300/AdMMP8 were approximately one-fifth the size of vehicle injected of control group tumors (508±158 vs. 2530±770) and one-third the size of Adwt300/AdCon injected group tumors (508±158 vs. 1436±627).

FIG. 6 shows in vivo mRNA expression. Total RNA extracted from the fresh tumor tissues was used as template for RT-PCR of MMP-8. Four of 6 mice tumors in Adwt300/AdMMP8 virus group were positive at 42 days from the time of injection, compared with 3 of 6 mice tumors in the Adwt300/Adcon group positive at day 26 when these animals with rapidly growing tumors were sacrificed. The positive control is A549 cells infected with AdMMP8 in vitro (lane 1), the negative control is from a vehicle injected tumor (lane 2). The expression of GAPDH is shown as a control.

FIG. 7 shows Masson's trichrome staining for collagen and immunohistochemistry for adenoviral capsid proteins in aligned serial sections. A549 xenograft tumors contained abundant and dense collagen bands staining blue in the vehicle injected control group (FIGS. 7A and B). In the Adwt300/AdCon, virus-injected tumors replicating adenoviral spread was inefficient (FIG. 7E), only a few cells in these tumors were infected based on immunohistochemistry for viral proteins (insert shows enlarged image) and abundant collagen bands were present in necrotic and surrounding areas (FIG. 7F). However, in the Adwt300/AdMMP8 injected tumors, collagen degradation was evident (FIG. 7D) and associated with extensive necrosis and the presence of adenovirus as detected by immunohistochemistry (FIG. 7C, many brown staining cells apparent in upper right quadrant of image, insert shows enlarged image). AdMMP8/AdCon injected tumors showed little evidence of collagen degradation (FIG. 7H) and no adenoviral staining (FIG. 7G).

FIG. 8 shows the amounts of collagen within necrotic areas. The amounts of collagen were scored by two blinded observers by ranking the intensity of collagen staining for all the tumors. A significant reduction in collagen within the Adwt300/AdMMP8 tumors compared to Adwt300/Adcon was seen (p<0.0001). The AdMMP8/Adcon group also scored with less collagen compared to the vehicle control group (p=0.0005) and Adwt300/Adcon group (<0.0001).

### DETAILED DESCRIPTION OF THE INVENTION

The present invention relates to a method of treating cancer in a subject. This involves co-administering a replicating virus and a matrix metalloproteinase to the subject under conditions effective to treat cancer.

The term “replicating virus” is meant to include a virus which undergoes the process of intracellular viral multiplication, consisting of the synthesis of proteins, nucleic acids, and sometimes lipids, and their assembly into a new infectious particle.

It is preferable that the replicating virus be a replicating adenovirus, reovirus, a replicating herpesvirus, vaccinia, measles, and vesicular stomatitis.

The replicating virus is preferably a replicating adenovirus. As used herein, the term “adenovirus” refers to any of a group of DNA-containing viruses (small infectious agents) that cause conjunctivitis and upper respiratory tract infections in humans. Adenoviral vectors are described in Peng, Z., “Current Status of Gendicine in China: Recombinant Human Ad-p53 Agent for Treatment of Cancers,” Hum Gene Ther 16:1016-1027 (2005); No authors listed, “The End of the Beginning: Oncolytic Virotherapy Achieves Clinical Proof-of-concept,” Mol Ther 13:237-238 (2006); Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003); Vile et al., “The Oncolytic Virotherapy Treatment Platform for Cancer: Unique Biological and Biosafety Points to Consider,” Cancer Gene Ther 9:1062-1067 (2002); Harrison et al., “Wild-type Adenovirus Decreases Tumor Xenograft Growth, but Despite Viral Persistence Complete Tumor Responses are Rarely Achieved—Deletion of the Viral E1b-19-kD Gene Increases the Viral Oncolytic Effect,” Hum Gene Ther 12:1323-1332 (2001); Kirn et al., “Clinical Research Results with dl1520 (Onyx-015), a Replication-selective Adenovirus for the Treatment of Cancer: What Have We Learned?,” Gene Ther 8:89-98 (2001); and Thorne et al., “Oncolytic Virotherapy: Approaches to Tumor Targeting and Enhancing Antitumor Effects,” Semin Oncol 32:537-548 (2005), which are hereby incorporated by reference in their entirety.

Other replicating viruses include reovirus and a replicating herpesvirus. As used herein, the term “reovirus” refers to a family of viruses that includes some viruses that affect the gastrointestinal system (such as Rotavirus), and some that cause respiratory infections. The genetic material of viruses in this family is double-stranded RNA. Reovirus infection occurs often in humans, but most cases are mild or subclinical. The virus can be readily detected in faeces, and many also be recovered from pharyngeal or nasal secretions, urine, cerebrospinal fluid, and blood. Despite the ease of finding reovirus in clinical specimens, their role in human disease or treatment is still uncertain. Reovirus is described in Wu et al, “Gene Transfer Facilitated by a Cellular Targeting Molecule, Reovirus Protein Sigma-1,” Gene Therapy 7:61-69 (2000); Wu et al., “M Cell-targeted DNA Vaccination,” PNAS 98(16):9318-9323 (2001); Beattie et al., “Reversal of the Interferon-Sensitive Phenotype of a Vaccinia Virus Lacking E3L by Expression of the Reovirus S4 Gene,” J Virol 69:499-505 (1995); and Imani et al., “Inhibitory Activity for the Interferon-induced Protein Kinase is Associated with the Reovirus Serotype 1 Sigma 3 Protein,” PNAS 85:7887-7891 (1988), which are hereby incorporated by reference in their entirety.

As used herein, the term “herpesvirus” refers to any of a group of DNA-containing animal viruses that form characteristic inclusion bodies within the nuclei of host cells and cause diseases such as chickenpox, infectious mononucleosis, herpes simplex virus (HSV), and shingles. Herpesvirus is described in Markert et al., “Oncolytic HSV-1 for the Treatment of Brain Tumours,” Herpes 13(3):66-71 (2006); Rainov et al., “Oncolytic Viruses for Treatment of Malignant Brain Tumours,” Acta Neurochir Suppl 88:113-23 (2003); Arvin A., “Investigations of the Pathogenesis of Varicella Zoster Virus Infection in the SCIDhu Mouse Model,” Herpes 13(3):75-80 (2006); Boeckh, M., “Prevention of VZV Infection in Immunosuppressed Patients Using Antiviral Agents,” Herpes 13(3):60-65 (2006); Mohr et al., “A Herpesvirus Genetic Element Which Affects Translation in the Absence of the Viral GADD34 Function,” EMBO J 15:4759-4766 (1996); and Honess et al., “Regulation of Herpesvirus Marcomolecular Synthesis, I. Cascade Regulation of the Synthesis of Three Groups of Viral Proteins,” J Virol 14:8-19 (1974), which are hereby incorporated by reference in their entirety.

As used herein, the term “matrix metalloproteinase” (MMP) refers to a member of a group of enzymes that can break down proteins, such as collagen, that are normally found in the spaces between cells in tissues (i.e., extracellular matrix proteins). Since these enzymes need zinc or calcium atoms to work properly, they are called metalloproteinases. Matrix metalloproteinases are involved in wound healing, angiogenesis, and tumor cell metastasis.

Such matrix-digesting enzymes are expressed during stages of normal embryogenesis, pregnancy, and other processes involving tissue remodelling. In addition, some of these enzymes, for example some matrix metalloproteinases (MMPs), degrade the large extracellular matrix proteins of the parenchymal and vascular basement membranes that serve as mechanical barriers to tumor cell migration. These MMPs are produced in certain cancers and are associated with metastasis (Liotta et al., Cell, 64:327-336 (1991), which is hereby incorporated by reference in its entirety). Examples of MMPs are the type IV collagenases, e.g., MMP-2 (gelatinase A. EC 3.4.24.24) and MMP-9 (gelatinase B, 3.4.24.35), and stromelysins (EC 3.4.24.17 and 3.4.24.22).

The MMP families appear of fundamental importance. The MMPs are endopeptidases that can cleave most components of the ECM. They were initially grouped based on substrate specificity, hence collagenases, gelatinases, stromelysins, and matrilysins. They are now numbered and grouped according to structure (Egeblad et al., “New Functions for the Matrix Metalloproteinases in Cancer Progression,” Nat Rev Cancer 2:161-174 (2002), which is hereby incorporated by reference in its entirety). The actions of this group of proteases can have profound effects on cancer-cell growth, differentiation, apoptosis, migration and invasion, and on the regulation of tumor angiogenesis and immune surveillance. In addition to cleaving structural ECM components the MMPs activate one another. They participate in the release of cell-membrane-bound precursor forms of many growth factors, including transforming growth factor-α, and TGF-β. Growth-factor receptors like FGF and HER2/neu are MMP substrates, as are cell-adhesion molecules like E-cadherin, CD44 and the αV-integrin subunit precursor. Modification of the cell adhesion molecules can lead to enhanced cancer cell invasion and released extracellular receptor domains may compete for activating ligands and thereby down-regulate receptor function (Egeblad et al., “New Functions for the Matrix Metalloproteinases in Cancer Progression,” Nat Rev Cancer 2:161-174 (2002), which is hereby incorporated by reference in its entirety).

The matrix metalloproteinases are identified as matrix metalloproteinase-1 to 28.

MMP-1 to MMP-28 are grouped as collagenases, gelatinases, stromelysins, and membrane type MMPs (MT-MMPs). The collagenases are capable of degrading triple-helical fibrillar collagens into distinctive ¾ and ¼ fragments. These collagens are the major components of bone and cartilage, and MMPs are the only known mammalian enzymes capable of degrading them. The collagenases are often referred to as MMPs: 1, 8, 13, and 18. In addition, MMP-14 has also been shown to cleave fibrillar collagen (Itoh et al., “Cell Surface Collagenolysis Requires Homodimerization of the Membrane-bound Collagenase MT1-MMP,” Molec Biol Cell 17:5390-5399 (2006), which is hereby incorporated by reference in its entirety). More controversially, there is evidence that MMP-2 is capable of collagenolysis (Karagiannis et al., “A Theoretical Model of Type I Collagen Proteolysis by Matrix Metalloproteinase (MMP) 2 and Membrane Type 1 MMP in the Presence of Tissue Inhibitor of Metalloproteinase 2,” J Biol Chem 279(37)39105-39114 (2004), which is hereby incorporated by reference in its entirety).

The main substrates of the gelatinases are type IV collagen and gelatin, and these enzymes are distinguished by the presence of an additional domain inserted into the catalytic domain. The gelatinases are MMPs 2 and 9 (Chakrabarti et al., “Matrix Metalloproteinase-2 (MMP-2) and MMP-9 in Pulmonary Pathology,” Exp Lung Res 31(6):599-621 (2005), which is hereby incorporated by reference in its entirety).

The stromelysins display a broad ability to cleave extracellular matrix proteins but are unable to cleave the triple-helical fibrillar collagens. Members of this group include MMPs 3, 10, and 11 (Hu et al., “Matrix Metalloproteinases and Their Tissue Inhibitors in the Developing Neonatal Mouse Uterus,” Biol Reprod 71:1598-1604 (2004), which is hereby incorporated by reference in its entirety).

The MT-MMPs constitute a growing subclass of recently identified matrix metalloproteinases and include MMPs 14, 15, 16, 17, 24, and 25 (Fillmore et al., “Membrane-type Matrix Metalloproteinases (MT-MMPs): Expression and Function During Glioma Invasion,” J Neurooncol 53(2):187-202 (2001), which is hereby incorporated by reference in its entirety).

Matrix metalloproteinase-8 (MMP-8), also known as collagenase-2 or neutrophil collagenase, is particularly preferred for use in the present invention, MMP-8 was long thought to be expressed solely by maturing neutrophils, and functionally restricted to ECM breakdown. Recent experiments, however, have revealed that this protease can be expressed by a wide variety of cell types and that it plays an important regulatory role in both acute and chronic inflammation (Van Lint et al, “Matrix Metalloproteinase-8: Cleavage can be Decisive, Cytokine Growth Factor Reviews 17(4)217-23 (2006), which is hereby incorporated by reference in its entirety).

The majority of the MMPs are known to exist in mammalian systems. The nucleic acid and amino acid sequences for MMP-1 to MMP-28 (with the exceptions of MMP-4, MMP-5, MMP-6, MMP-18 (no known human orthologue), and MMP-22) may be found using the following reference sequence ID numbers on GenBank: MMP-1 (NM_{—}002421), MMP-2 (NM_{—}004530), MMP-3 (NM_{—}002422), MMP-7 (NM_{—}002423), of particular interest MMP-8 (NM_{—}002424), MMP-9 (NM_{—}004994), MMP-10 (NM_{—}002425), MMP-11 (NM_{—}005940), MMP-12 (NM_{—}002426), MMP-13 (NM_{—}002427), MMP-14 (NM_{—}004995), MMP-15 (NM_{—}002428), MMP-16 (NM_{—}005941), MMP-17 (NM_{—}016155), MMP-19 (NM_{—}002429), MMP-20 (NM_{—}004771), MMP-21 (NM_{—}147191), MMP-23 (A-NR_{—}002946, B-NM_{—}006983), MMP-24 (NM_{—}006690), MMP-25 (NM_{—}022468), MMP-26 (NM_{—}021801), MMP-27 (NM_{—}022122), and MMP-28 (NM_{—}024302). These sequences are hereby incorporated by reference in their entirety.

One embodiment of the present invention relates to a method of administering the matrix metalloproteinase as a nucleic acid encoding the matrix metalloproteinase which is operatively positioned in the replicating virus under conditions effective to express the matrix metalloproteinase.

As noted above, viral vectors have been successfully employed in order to increase the efficiency of introducing a recombinant vector into suitably sensitive host cells. Therefore, viral vectors are particularly suited for use in the present invention. Current research in the field of viral vectors is producing improved viral vectors with high-titer and high-efficiency of transduction in mammalian cells (see, e.g., U.S. Pat. No. 6,218,187 to Finer et al., which is hereby incorporated by reference in its entirety). Such vectors are suitable in the present invention, as is any viral vector that comprises a combination of desirable elements derived from one or more of the viral vectors described herein. It is not intended that the expression vector be limited to a particular viral vector.

Certain “control elements” or “regulatory sequences” are also incorporated into the vector-construct. The term “control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription, and translation of a coding sequence in a recipient cell. Not all of these control elements need always be present so long as the selected coding sequence is capable of being replicated, transcribed, and translated in an appropriate host cell.

The term “promoter region” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcriptional control signals in eukaryotes comprise “promoter” and “enhancer” elements. Promoter and enhancer elements have been isolated from a variety of eukaryotic sources, including genes in yeast, insect, and mammalian cells, and viruses. Analogous control elements, i.e., promoters, are also found in prokaryotes. Such elements may vary in their strength and specificity. For example, promoters may be “constitutive” or “inducible.”

A constitutive promoter is a promoter that directs expression of a gene throughout the development and life of an organism. Examples of some constitutive promoters that are widely used for inducing expression of transgenes include the nopoline synthase (NOS) gene promoter from Agrobacterium tumefaciens (U.S. Pat. No. 5,034,322 to Rogers et al., which is hereby incorporated by reference in its entirety), the cytomegalovirus (CMV) early promoter, those derived from any of the several actin genes, which are known to be expressed in most cells types (U.S. Pat. No. 6,002,068 to Privalle et al., which is hereby incorporated by reference in its entirety), and the ubiquitin promoter, which is a gene product known to accumulate in many cell types.

An inducible promoter is a promoter that is capable of directly or indirectly activating transcription of one or more DNA sequences or genes in response to an inducer. In the absence of an inducer, the DNA sequences or genes will not be transcribed. The inducer can be a chemical agent, such as a metabolite, or a physiological stress directly imposed upon the organism such as cold, heat, toxins, or through the action of a pathogen or disease agent. A recombinant cell containing an inducible promoter may be exposed to an inducer by externally applying the inducer to the cell or organism by exposure to the appropriate environmental condition or the operative pathogen.

Inducible promoters may be used in the viral vectors of this invention. These promoters will initiate transcription only in the presence of an additional molecule. Examples of inducible promoters include the tetracycline response element and promoters derived from the β-interferon gene, heat shock gene, metallothionein gene or any obtainable from steroid hormone-responsive genes. Tissue specific expression has been well characterized in the field of gene expression and tissue specific and inducible promoters are well known in the art. These genes are used to regulate the expression of the foreign gene after it has been introduced into the target cell.

To ensure efficient expression, 3′ polyadenylation regions must be present to provide for proper maturation of the mRNA transcripts. The native 3′-untranslated region of the gene of interest is preferably used, but the polyadenylation signal from, for example, SV40, particularly including a splice site, which provides for more efficient expression, could also be used. Alternatively, the 3′-untranslated region derived from a gene highly expressed in a particular cell type could be fused with the gene of interest.

According to another embodiment, the matrix metalloproteinase is administered as a nucleic acid encoding the matrix metalloproteinase, and the nucleic acid is operatively positioned in a non-replicating virus. The term “non-replicating virus” refers to vaccines which are made by deleting one or more genes from a virus capable of entering human cells. Using HIV as an example, in place of the deleted genes are inserted segments of DNA encoding HIV proteins (transgenes), resulting in a replication-incompetent viral vector. The vector carries the HIV transgenes into cells, where they are expressed as HIV peptides or proteins. The non-replicating virus is selected from the group consisting of adeno-associated virus, a non-replicating adenovirus, and a non-replicating herpesvirus.

The matrix metalloproteinase may alternatively be administered as a protein.

According to one embodiment, the cancer is selected from the group consisting of lung cancer, colon cancer, breast cancer, prostate cancer, pancreatic cancer, and glioma cancer. The subject can be a human.

The therapeutic of the present invention may be administered to a subject, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

To improve the efficacy of the present invention, the therapeutic of the present invention is administered with an adjuvant. Suitable adjuvants include aluminum salts (alum) such as aluminum hydroxide, aluminum phosphate, and aluminum sulfate, Incomplete Feund's Adjuvant (IFA), and monophosphoryllipid A (MPL). These adjuvants are suitable for human administration, either alone or optionally all combinations thereof (Chang et al., “Adjuvant Activity of Incomplete Freund's Adjuvant,” Adv Drug Deliv Rev 32:173-186 (1998), which is hereby incorporated by reference in its entirety). Other adjuvants include cytokines, such as interleukins (IL-1, IL-2, and IL-12), macrophage colony stimulating factor (M-CSF), and tumor necrosis factor (TNF).

The therapeutic of the present invention can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The therapeutic of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, the therapeutic may be incorporated with excipients and used in the form of tablets, capsules, elixirs, suspensions, syrups, and the like. Various other materials may be present as coatings or to modify the physical form of the dosage unit.

The therapeutic may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The therapeutic of the present invention may also be administered directly to the airways in the form of an aerosol. For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

The therapeutic of this invention may be administered in sufficient amounts to transfect the desired cells and provide sufficient levels of integration and expression of the replicating virus to provide a therapeutic benefit without undue adverse effects or with medically acceptable physiological effects which can be determined by those skilled in the medical arts.

Dosages of the therapeutic will depend primarily on factors, such as the condition being treated, the age, weight, and health of the patient, and may thus vary among patients. The dosage will be adjusted to balance the therapeutic benefit against any viral toxicity or side effects.

The present invention also relates to a method of enhancing the delivery to and distribution within a tumor mass of therapeutic viruses. This involves co-administering a replicating virus and a matrix metalloproteinase to the tumor mass under conditions effective to enhance the delivery to and distribution within the tumor mass of therapeutic viruses.

This aspect of the present invention can be carried out by formulating and administering the vectors matrix metalloproteinases in substantially the same manner as described above.

Another aspect of the present invention relates to a cancer therapeutic. This involves a replicating virus and a matrix metalloproteinase.

This aspect of the present invention can be formulated as described above.

### EXAMPLES

The following examples are provided to illustrate embodiments of the present invention but are by no means intended to limit its scope.

### Materials and Methods

### Examples 1-6

### Cell Lines

A549 lung adenocarcinoma cells (American Type Culture Collection, Rockville, Md.; ATCC, CCL 185) were cultured in F1K medium with 10% fetal bovine serum (FBS), penicillin, and streptomycin. A375 melanoma cell (ATCC, CRL 1619) were cultured in Dulbecco's Modified Eagle's Medium with 4 mM L-glutamine, 4.5 g/L glucose, 1.5 g/L sodium bicarbonate and antibiotics. 293 cells (Human embryonic kidney cells transformed by human adenovirus type 5, Toronto, Ontario, Canada; Microbix) were cultured in Eagle's minimal essential medium (MEM) with 10% FBS and antibiotics.

### Total RNA Isolation and cDNA Synthesis

Total RNA was extracted from cells or tissues by using Micro-to-Midi Total RNA Purification System (Invitrogen, Carlsbad, Calif.). Genomic DNA was removed by RQ1 RNase-Free DNase treatment (Promega, Madison, Wis.). mRNA was reverse transcribed using AMV reverse transcriptase and oligo(dT)₁₅ primers from Universal RiboClone cDNA Synthesis System (Promega). PCR primers were designed based on the human MMP-8 cDNA sequence (GenBank Accession number NM_{—}002424), to specifically amplify full-length mRNA, forward primer 5′ AAAGAAAGCCAGGAGGGGTA 3′ (SEQ ID NO: 1), reverse primer 5′ CGGAGGACAGGTAGAATGGA 3′ (SEQ ID NO: 2), predicted size of cDNA amplification product is 1585 bp, coding sequence is 1401 bp. A second pair of forward and reverse primers will produce a 639 bp fragment, which was used for semi-quantitative reverse transcription PCR, forward primer 5′ ATCTCACAGGGAGAGGCAGA 3′ (SEQ ID NO: 3), reverse primer 3′ CCTTGGGATAACCTTGCAGA 3′ (SEQ ID NO: 4). The expression of MMP-8 gene was normalized to glyceradehyde-3-phosphate dehydrogenase (GAPDH) gene expression.

### Adenoviruses and Recombinant Adenoviral Construction

The wild type virus used in these experiments was Adwt300. Adβ-gal is a non-replicating adenovirus expressing the β-gal transgene. AdMMP-8, an E1 and E3 gene deleted adenoviral DNA vector expressing the human MMP-8 under the control of the cytomegalovirus promoter (CMV) with a simian virus 40 polyadenylation signal, was constructed using the AdEasy™ XL Adenoviral Vector System (Stratagene, La Jolla, Calif.). The human MMP-8 full-length cDNA product was ligated into pcDNA3.1/V5-His-TOPO-TA Expression Vector (Invitrogen), and the sequence confirmed. In the AdEasy XL system, the human MMP-8 cDNA was cloned into pShuttle-CMV, once constructed the shuttle vector was linearized with PmeI and co-transformed into E. coli BJ5183 electroporation competent cells along with the adenoviral backbone vector. Electroporation was performed in 2.0 mm cuvettes at 2500V, 200 ohms and 25 micro-FD in a Bio-Rad Gene Pulser electroporator. Transformants were selected for kanamycin resistance, and recombinants subsequently identified by restriction digestion. Purified recombinant Ad plasmid DNA was digested with PacI to expose its inverted terminal repeats (ITRs), and was then used to transfect 293 cells for large-scale preparation. Adcon, a control non-replicating virus without a transgene was constructed in the same way.

### Immunoblotting

A549 cells were infected with AdMMP8 at a multiplicity of infection of (MOI) 200 for 24 h. To prepare serum-free conditioned medium, cells were washed six times with serum-free medium and then resuspended in fresh serum-free medium. After 24 h, the culture supernatant was aspirated, spun at 1,500×g to remove cellular debris, and concentrated approximately 100-fold using Biomax Ultrafree Centrifugal Filters (Millipore, Bedford, Mass.). Total protein concentration was determined by the bicinchoninic acid (BCA) protein assay (Pierce, Rockford, Ill.), and equal amounts of denatured and reduced protein were separated on a 10% SDS-PAGE glycine gel, and proteins were transferred onto a polyvinylidene difluoride (PVDF) membrane (Immobilon-P, Millipore, Bedford, Mass.). The membranes were blocked in phosphate-buffered saline with 0.1% Tween 20 and 5% non-fat dried milk for 2 h at room temperature, followed by incubation with goat polyclonal anti-MMP-8 primary antibody antibody (Santa Cruz Biotechnology) at 1:500 dilution overnight at 4° C. After washing with phosphate-buffered saline-Tween 20 four times, membranes were incubated with horseradish peroxidase-conjugated donkey anti-goat secondary antibody (Santa Cruz Biotechnology) for 1 h at room temperature. Antibody binding was visualized by enhanced chemiluminescence (Supersignal West Pico Chemiluminescent Substrate, Pierce, Rockford, Ill.).

### Zymography

The analyses were performed as has been previously described (Montel et al., “Altered Metastatic Behavior of Human Breast Cancer Cells After Experimental Manipulation of Matrix Metalloproteinase 8 Gene Expression,” Cancer Res 64:1687-1694 (2004), which is hereby incorporated by reference in its entirety). Briefly, 25 μg of unheated protein samples from concentrated serum-free conditional medium were loaded on 10% Tris-glycine polyacrylamide gel with 0.1% gelatin incorporated as a substrate (Bio-Rad Life Science, Hercules, Calif.). After a room temperature migration under non-reducing conditions, the gel was incubated twice for 15 min at room temperature in renaturing buffer (Bio-Rad Life Science), equilibrating for 30 min at room temperature in the developing buffer and incubated overnight at 37° C. in fresh developing buffer. The gel was stained with 0.5% Coomassie Brilliant Blue R in 50% methanol/10% acetic acid for 30 min and destained in 7.5% acetic acid/5% methanol. The clear bands represent gelatinase activity.

### Diffusion Assays

Virus diffusion through matrix components was evaluated using BD Biocoat™ Cell Culture Inserts Variety Packs and Companion Plates (BD Bioscience, Discovery Labware, San Diego, Calif.). Matrix components pre-coated on 3 μm and 0.45 μm membranes included collagen I, collagen IV, fibronectin, laminin, and control. Briefly, 293 cells were plated on the base of 24 well plates (1.5×10⁵ cells per well) in 0.3 ml of MEM medium with 10% FBS. After 24 h incubation, an insert containing Adβ-gal virus (MOI 5) diluted in 0.3 ml of MEM medium with 10% FBS was placed onto the well. After 24 h infection, the ability of the virus to spread through the insert was analyzed by in situ staining of cells for β-galactosidase and by β-galactosidase assay. In an additional experiment, the collagen I insert was treated with collagenase from clostridium histolyticum (100 μg/ml) in MEM medium for 2 h at 37° C., and then washed three times with PBS buffer before application of the virus.

Albumin diffusion through matrix components was evaluated using the same inserts as above with the addition of a fibrillar collagen insert (BD Bioscience). Briefly, an insert containing bovine serum albumin 2 mg/ml in 0.35 ml was placed onto the 24 well plate, 0.8 ml PBS was plated on the base of well. At 24 h, 48 h and 72 h time points, the albumin concentration of both inserts and wells was measured by BCA Protein Assay.

To determine the effects of MMP-8 on fibrillar collagen inserts, A549 cells were seeded on the fibrillar collagen membrane and infected with AdMMP8 or control Adcon. After 7 days, wells were rinsed with PBS, and viral diffusion performed as previously described.

### In Situ Staining of Cells for β-Galactosidase Activity

The cell monolayers were rinsed twice in 2 ml of room temperature PBS, and then fixed in 0.5 ml of 0.5% glutaraldehyde solution for 15 min at room temperature. The glutaraldehyde was removed followed by rinsing 3 times in PBS, 5 min each time. Staining solution (0.5 ml of 100 mm potassium ferricyanide, 100 mm potassium ferriocynide, 200 mm MgCl₂, X-Gal 50 mg/ml in PBS) was added and the cells incubate at 37° C. for 3 h until the control cells were visibly stained.

### β-Galactosidase Assay

A β-Galactosidase Enzyme Assay System with Reporter Lysis Buffer (Promega) was used. The growth medium was removed from the cells, and the cells washed twice with PBS buffer. Reporter lysis buffer (200 μl) was then added to each well of the 24 well plates and incubated at room temperature for 15 min; the cell lysates were centrifuged at 14,000 rpm for 2 min at 4° C. The supernatants were then transferred to a fresh tube, and 30 μl of lysate was mixed with 20 μl of reporter lysis buffer. The diluted cell lysates were added to the wells of a 96 well plate, plus 50 μl of assay buffer and incubated at 37° C. for 30 min. The reaction was stopped by adding 150 μl of 1M sodium carbonate, and the absorbance of the samples read at 420 nm in a plate reader.

### Established Tumor Xenograft Model

A549 cells (5×10⁶) were injected into the right flank of NCrNU-M nude mice (Taconic, Germantown, N.Y.) that were 6-7 weeks of age. Tumor size (length and breadth) was measured twice weekly, and tumor volume was calculated on the basis of the following formula: length(breadth/2)²π. Virus was injected when the tumors reached approximately 250 mm³ in size. A total of 1×10⁹ PFU of equal amounts of two viruses were injected in a total volume of 50 μl split into the four quadrants of the tumors. The viral combinations were as follows: Adwt300 plus nonreplicating AdMMMP8; Adwt300 plus nonreplicating Adcon vector; AdMMP8 plus Adcon vector; vehicle control. Animals were killed at day 42 or before if tumor volumes were estimated to exceed 10% of the animals' body weight.

### Histology and Immunohistochemistry

Tumor tissue was fixed in formalin (10%), embedded in paraffin and cut into 5-μm sections (Wax-it Histology Services, Vancouver, Canada). Representative sections were stained with hematoxylin and eosin (H&E) and examined by light microscopy. Masson's trichrome staining for collagen was performed using aniline blue (Wax-it Histology Services). For immunohistochemistry, slides were deparaffinized in xylene and then hydrated through graded alcohol. Adenovirus antigen detection was performed using methods and reagents supplied by DAKO (“DAKO ARK”, Dako, Carpinteria, Calif.). Endogenous peroxidase in the section was blocked with hydrogen peroxide (0.03%). The primary mouse antiadenovirus antibody (MAB8052, Chemicon, Temecula, Calif.) was first complexed with a biotinylated anti-mouse secondary antibody (DAKO). Mouse serum was added to minimize potential interaction between uncomplexed secondary antibody and mouse immunoglobulin in the tissue section. The antibody complex was then applied to the sections and incubated 15 min, rinsed in buffer, and incubated in streptavidin-peroxidase. Diaminobenzidine/hydrogen peroxidase was used to as the chromogen substance.

### Statistical Analyses

Data are reported as mean±standard error of the mean. Analysis of variance with post hoc testing was used to compare multiple groups. For the animal experiments Kaplan-Meier survival curves were generated based on the time to tumor trebling after viral administration. Survival curves were compared using a log-rank test.

### Example 1

### Effect of Matrix Components on Adenoviral Diffusion

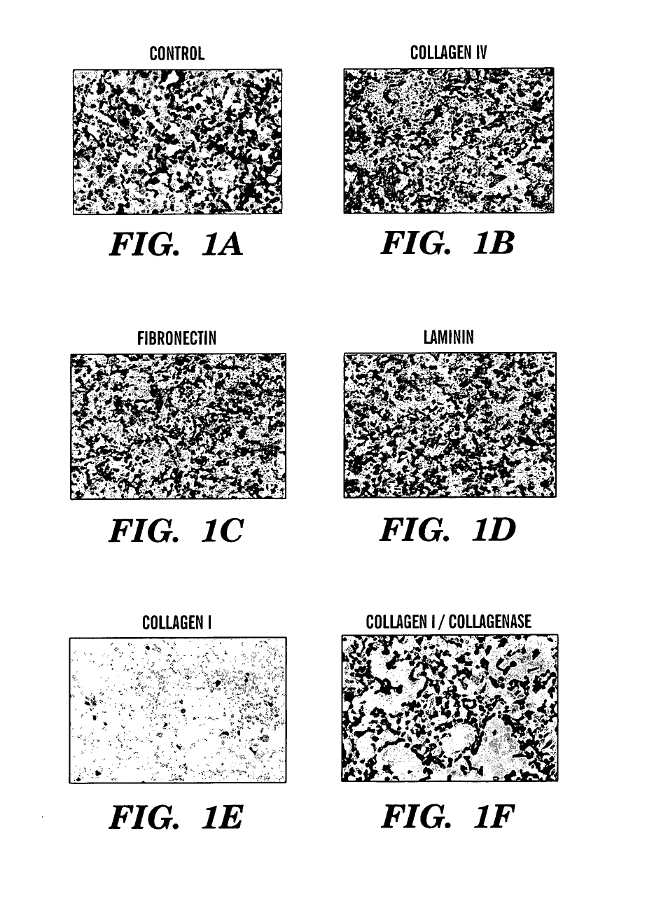

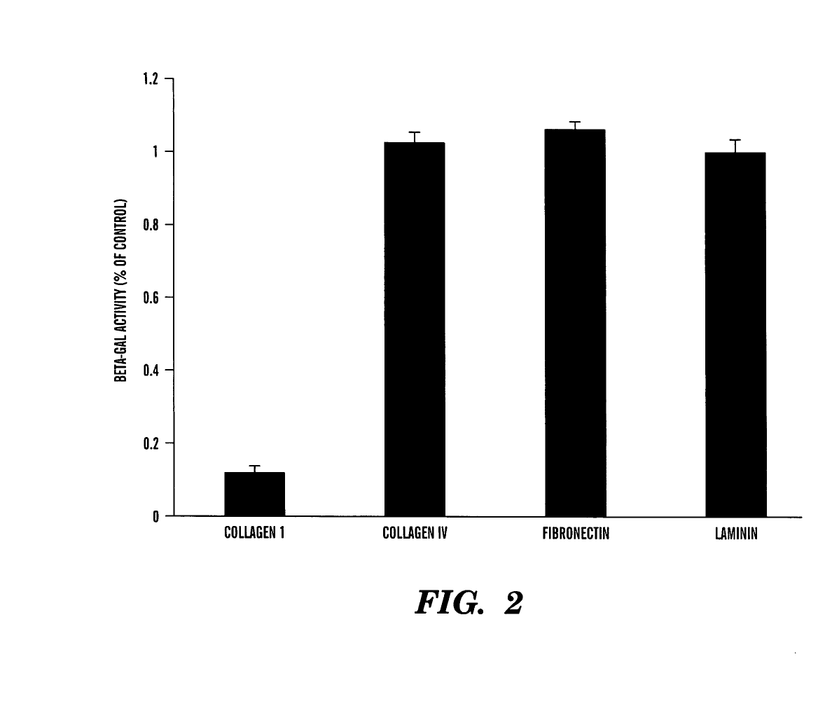

The matrix components that were examined in this experiment include collagen I, collagen IV, fibronectin, and laminin. These matrix components were obtained commercially pre-coated on membrane inserts. The membrane inserts were placed into the chambers of 24-well plates that had been pre-seeded with 293 cells growing as a monolayer on the base. Adβ-gal, an E1a-deleted β-galactosidase gene expressing virus, was added to the upper chamber. To infect the 293 cells plated on the base of the 24 well plates, the virus had to spread through the matrix-coated insert. Only Adβ-gal virus infected 293 cells would be expected to express β-galactosidase. The collagen I insert blocked the spread of Adβ-gal virus so that only a few 293 cells stained blue after the addition of X-gal. In contrast, control non-coated, collagen IV, fibronectin, and laminin inserts allowed the passage of virus with subsequent intense blue staining of the 293 indicator cells as shown in FIG. 1. Collagen 1 markedly reduces the proportion of cells expressing β-gal (FIG. 1E) compared to control (FIG. 1A) and other matrix components (FIG. 1B-D), and this can be restored to control levels by pre-treatment with collagenase (FIG. 1F). This experiment was repeated, but instead of X-gal staining of the monolayer, a quantitative β-galactosidase assay was performed on lysates of the 293 cells. The β-galactosidase activity was significantly decreased in the cell lysates obtained from the collagen I plates (p<0.0001) compared to the collagen IV, fibronectin, and laminin insert plates that were not different from control as shown in FIG. 2. Similar results were obtained with 3.0 μm and 0.45 μm membrane pore sizes (except collagen IV was not performed on a 0.45 μm membrane).

To further determine if collagen I plays an important role in the regulation of diffusion of adenovirus, collagen I inserts were treated with collagenase. Collagenase treatment of the collagen I insert restored the ability of Adβ-gal to diffuse and infect the 293 indicator cells. The 293 cells were intensely stained blue compared with the untreated collagen I insert in which only a few cells stained blue, as shown in FIG. 1.

To determine if the virus bound to the collagen I, the Adβ-gal virus was placed onto the collagen I insert as previously, but then after 24 hours was aspirated and applied directly onto the 293 indicator cells. Similar proportions of positive blue cells were seen when taking the medium containing Adβ-gal virus from the collagen I insert upper compartment and infecting 293 cells compared with the same amount of Adβ-gal virus applied to and aspirated from the control insert. These data suggest that adenoviruses do not have a strong binding affinity to collagen, but that the collagen matrix serves as a physical barrier.

Tumor stroma is largely composed of collagen I, but the physical form within the tumor may be different than on the inserts. To evaluate the potential importance of collagen I in reducing viral spread in tumors in vivo, an adenoviral vector was constructed that expresses a metalloproteinase with the potential to breakdown collagen I.

### Example 2

### Expression of Functional MMP-8 by a Non-replicating Adenovirus

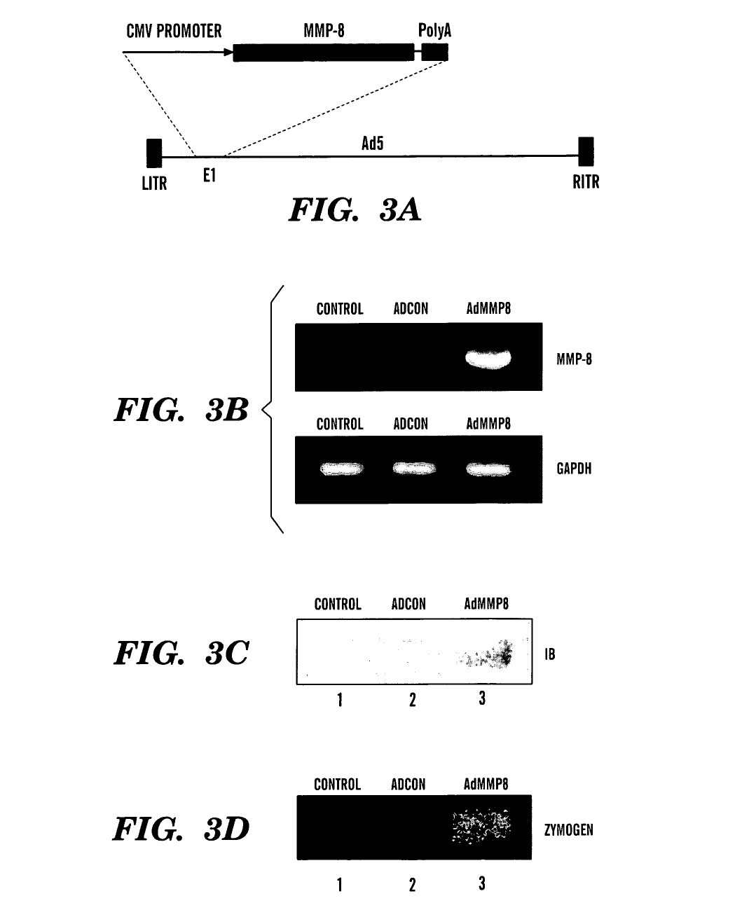

Based on reports of the effective collagen I-degrading activity of MMP-8 (Hasty et al., “The Collagen Substrate Specificity of Human Neutrophil Collagenase,” J Biol Chem 262:10048-10052 (1987), which is hereby incorporated by reference in its entirety), an adenovirus to express the MMP-8 transgene was constructed. As described in the methods section, the MMP-8 cDNA was cloned by PCR from RNA obtained from the A375 human melanoma cell line and inserted into the shuttle plasmid pCMV-shuttle. AdMMP8, a non-replicating adenoviral vector was constructed by homologous recombination in bacteria using the shuttle plasmid and the Adeasy viral genome plasmid, shown in FIG. 3A. In the recombinant AdMMP8 adenovirus, a CMV promoter drives the MMP-8 cDNA. A control vector without a transgene, Adcon, was constructed in a similar manner.

To investigate if the AdMMP8 adenovirus directed the expression of functional MMP-8 protein, lung A549 cancer cells were infected with recombinant AdMMP8. Native MMP-8 has a very limited distribution with expression restricted to a few tissues, namely neutrophils, chondrocytes, and some melanoma cell lines. Total RNA was extracted from AdMMP8 infected, control virus (Adcon) infected, and uninfected A549 cells and used as template for RT-PCR. Control A549 cells and Adcon infected A549 cells did not express MMP-8 mRNA, whereas AdMMP8 infected A549 cells expressed MMP-8 mRNA as demonstrated by a PCR product of the appropriate size, shown in FIG. 3B. Immunoblotting was also performed on the serum-free conditioned supernatants from the A549 cells under similar experimental conditions. The blots show a single band recognized by the polyclonal anti-MMP-8 antibody at 65 kDa in size (FIG. 3C). The collagenase activity of the supernatant was also demonstrated by zymography (FIG. 3D). The gelatin hydrolysis visible in the zymogram gel indicates that the recombinant MMP-8 generated by infection of A549 cells with AdMMP8 was not only secreted into the culture medium but was also functional on the gelatin substrate.

### Example 3

### A549 Cells Infected with AdMMP8 Break Down a Fibrillar Collagen Matrix

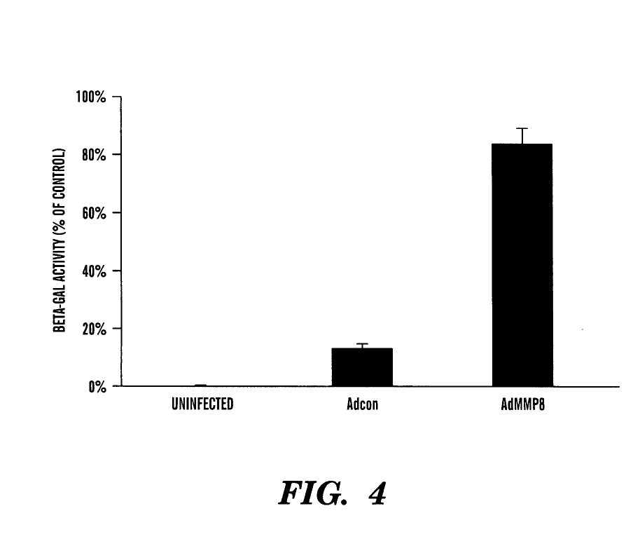

In this experiment, a fibrillar collagen matrix insert comprised of large collagen fibers in a lattice was used (Swiderek et al., “Effects of ECM Protein on Barrier Formation in Caco-2 Cells,” Becton Dickinson Technical Bulletin #421 (1997), which is hereby incorporated by reference in its entirety), because this resembles the tumor architecture more closely than amorphous collagen. Further, the tumor content of fibrillar collagen has been shown to influence diffusive transport within tumors (Brown et al., “Dynamic Imaging of Collagen and its Modulation in Tumors In Vivo Using Second-harmonic Generation,” Nat Med 9:796-800 (2003), which is hereby incorporated by reference in its entirety). To establish a potential role for MMP-8 in degrading fibrillar collagen, a fibrillar collagen membrane was conditioned by a monolayer of A549 cells infected with AdMMP8. The collagen membrane conditioned by Ad-MMP8 infected cells but not cells infected with a control virus became permeable to the adenovirus (FIG. 4).

The in vitro forms of collagen appear to form a physical barrier to viral diffusion. To evaluate this physical property albumin diffusion was studied. An albumin gradient of 400 was established (insert to well), this was reduced to a gradient of less than four by 24 hr for the collagen IV, fibronectin, and laminin and control membranes, but took 72 hrs to fall to a gradient of six for collagen I and to a gradient of 16 for fibrillar collagen membranes, by which time the other membranes were fully equilibrated. These experiments suggest that impaired viral transport results from the physical properties of the matrix components.

### Example 4

### MMP-8 Expression from AdMMP8 Improves the Oncolytic Efficacy of a Replicating Adenovirus

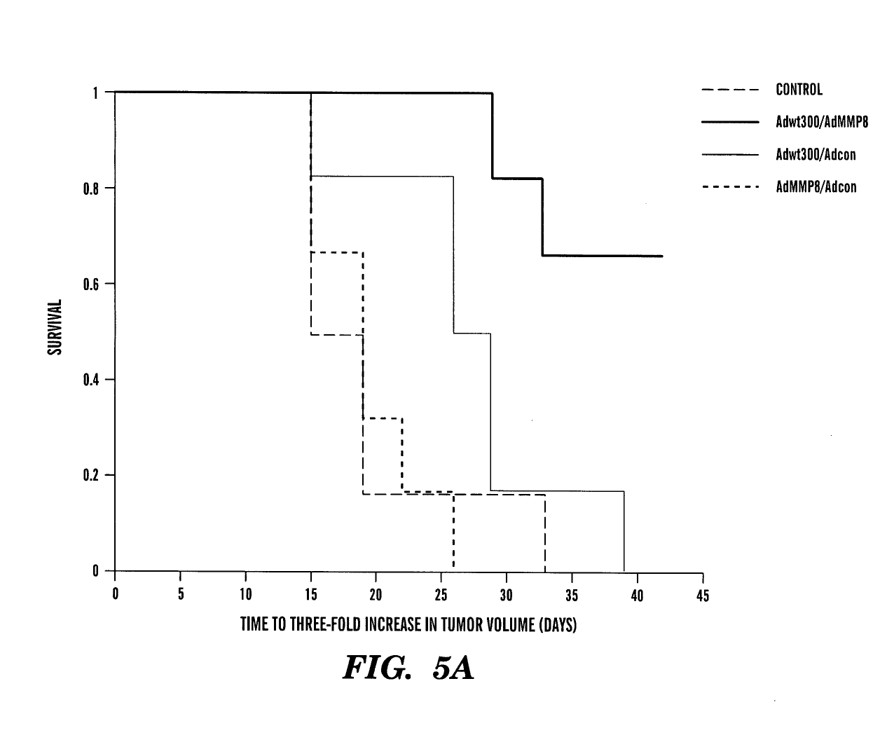

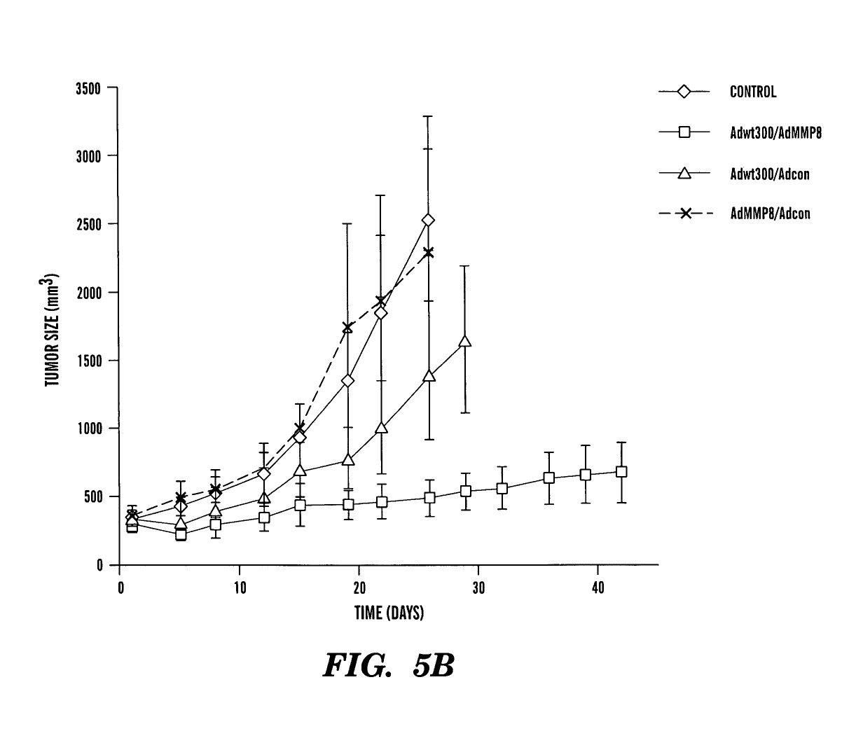

Previous studies have shown limited intratumoral spread of replicating adenoviral vectors in xenograft models; and based on the in vitro studies just described, matrix components, in particular collagen I, may present a hurdle for effective adenoviral spread. To determine whether MMP-8 expression within the tumor would improve the efficacy of the replicating adenovirus, established human lung A549 xenografts were injected with a wild-type replicating adenovirus Adwt300 (5×10⁸ PFU) together with the non-replicating AdMMP8 virus (5×10⁸ PFU). The addition of AdMMP8 to the wild type virus significantly increased the survival (survival endpoint defined as a three-fold increase in tumor size) of animals compared with control tumors injected with the wild type virus in combination with Adcon (p=0.008) as shown in FIG. 5A. At day 19, Adwt300/AdMMP8 group tumors were one-third the size of vehicle treated control group tumors (455±113 vs. 1354±358) and approximately half the size of Adwt300/Adcon group tumors (455±113 vs. 788±228) as shown in FIG. 5B.

AdMMP8 injection alone did not affect the growth of the lung A549 tumor xenografts. Tumor xenografts in mice treated with AdMMP8 (plus control Adcon virus) showed similar growth kinetics as vehicle treated controls. These results demonstrate that intratumoral AdMMP8 injection has no effect on the growth of lung A549 cell tumor xenografts. However, intratumoral AdMMP8 injection enhances the oncolytic effect of a replicating virus.

Lungs were evaluated in all groups for the presence of metastases, and none were visible.

### Example 5

### MMP-8 mRNA Expression Persists for 42 Days

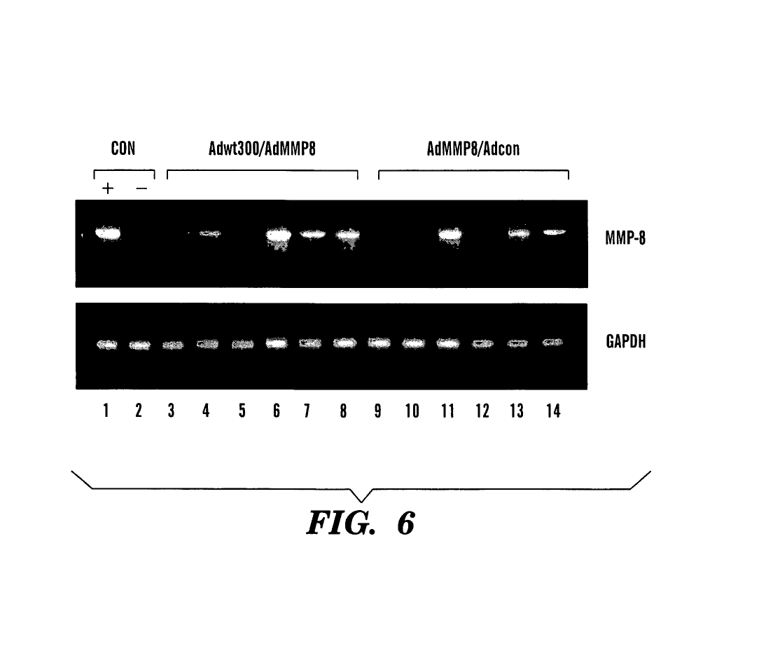

To confirm MMP-8 expression following viral administration, total RNA was extracted from the fresh tumor tissues at the time of sacrifice, and was used as a template for RT-PCR for MMP-8 mRNA. Four of six tumors in the Adwt300/AdMMP8 virus group were positive for MMP-8 mRNA at 42 days, and three of six tumors in the AdMMP-8/Adcon group at day 26 (FIG. 6). When the AdMMP8 non-replicating virus is co-infected with replicating Adwt300 virus, AdMMP8 might be anticipated to replicate as E1a may be provided by the replicating virus in trans (Thome et al., “Selective Intratumoral Amplification of an Antiangiogenic Vector by an Oncolytic Virus Produces Enhanced Antivascular and Anti-tumor Efficacy,” Mol Ther 13:938-946 (2006), which is hereby incorporated by reference in its entirety). Patchy distribution of the virus and sampling may explain the negative results for two tumors in the wt300/AdMMP-8 virus group, which incidentally were also the largest and least responsive tumors of the group.

### Example 6

### Improved Tumor Responses Induced by AdMMP8 in Combination with Wild Type Virus are Associated with Extensive Necrosis and Reduced Tumor Collagen Expression

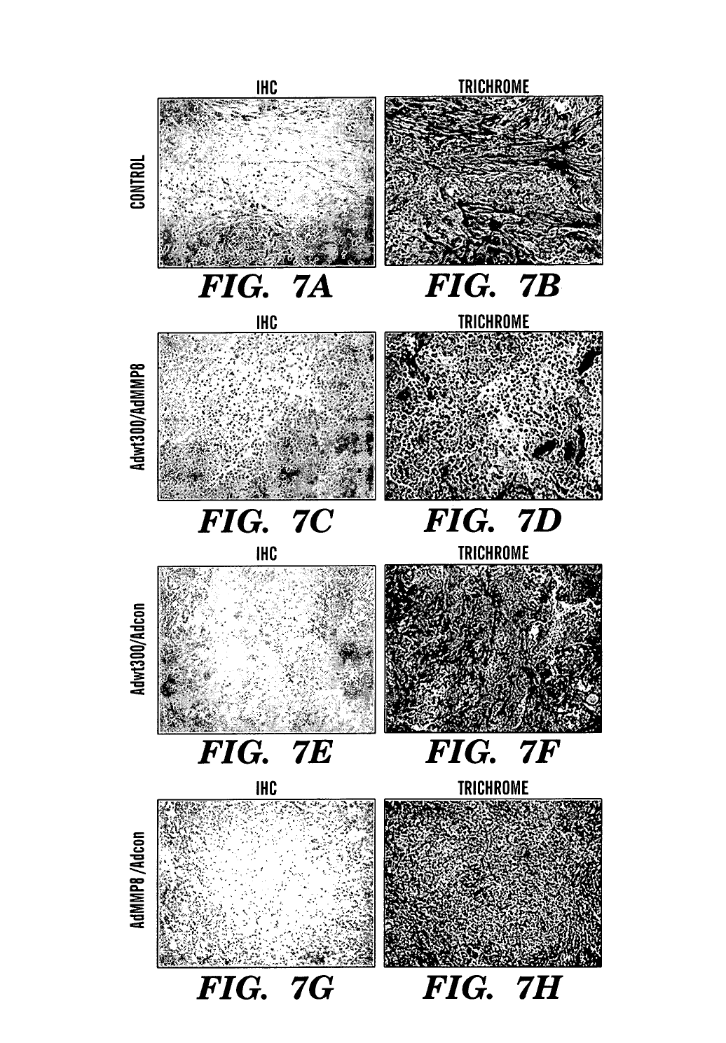

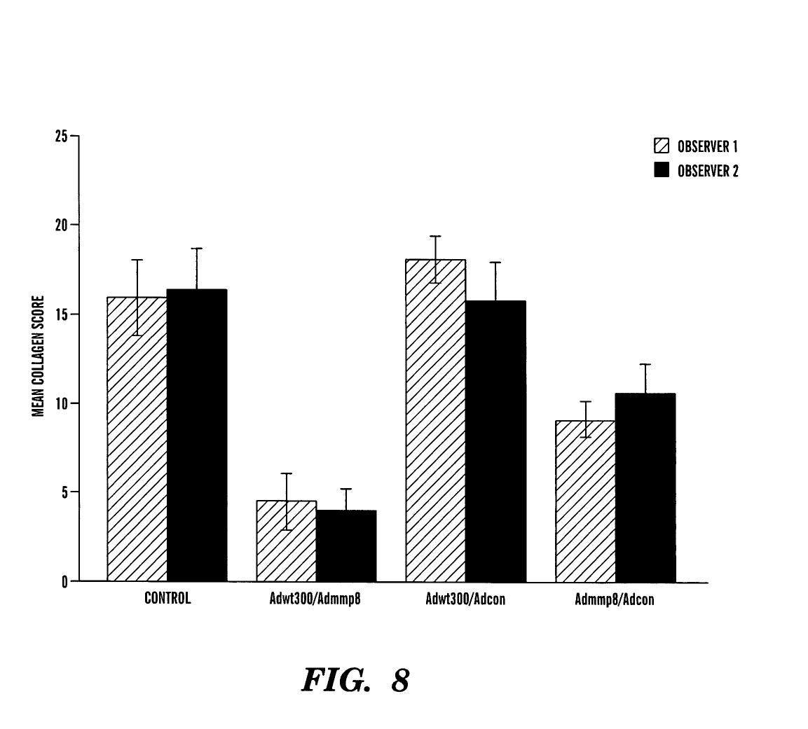

Virus distribution within the tumor and collagen expression was evaluated with Masson's trichrome staining for collagen and immunohistochemistry for adenoviral capsid proteins (FIG. 7). A549 xenograft tumors contained abundant and dense collagen bands in the vehicle injected control group tumors. In FIG. 7B, Adwt300 virus-injected tumors adenoviral spread was inefficient and patchy (FIG. 7E), and abundant collagen bands persisted in necrotic virus infected and surrounding areas (FIG. 7F). In contrast, in Adwt300 plus AdMMP8 injected tumors collagen degradation was more extensive (FIG. 7D) within the virus induced necrotic areas. The amounts of collagen within necrotic areas were scored by two blinded observers by ranking the intensity of collagen staining for all the tumors (FIG. 8). A significant reduction in collagen within the Adwt300/AdMMP8 tumors compared to Adwt300/Adcon was seen (p<0.0001). The AdMMP8/Adcon group also scored with less collagen compared to the vehicle control group (p=0.0005) and Adwt300/Adcon group (<0.0001).

Clinical experience and animal models using replicating adenoviral vectors indicate surprisingly poor intratumoral spread of the replicating virus (Sauthoff et al., “Intratumoral Spread of Wild-type Adenovirus is Limited After Local Injection of Human Xenograft Tumors: Virus Persists and Spreads Systematically at Late Time Points,” Hum Gene Ther 14:425-433 (2003); Vile et al., “The Oncolytic Virotherapy Treatment Platform for Cancer: Unique Biological and Biosafety Points to Consider,” Cancer Gene Ther 9:1062-1067 (2002); Harrison et al., “Wild-type Adenovirus Decreases Tumor Xenograft Growth, but Despite Viral Persistence Complete Tumor Responses are Rarely Achieved—Deletion of the Viral E1b-19-kD Gene Increases the Viral Oncolytic Effect,” Hum Gene Ther 12:1323-1332(2001); Kim et al., “Clinical Research Results with dl1520 (Onyx-015), a Replication-selective Adenovirus for the Treatment of Cancer: What Have We Learned?,” Gene Ther 8:89-98 (2001); Thorne et al., “Oncolytic Virotherapy: Approaches to Tumor Targeting and Enhancing Antitumor Effects,” Semin Oncol 32:537-548 (2005), which are hereby incorporated by reference in their entirety). Effective oncolytic virotherapy is likely to be dependent on more efficient and dispersed delivery of the replicating adenovirus to the tumor mass than is currently achieved. The causes of poor viral spread within tumors, even in immune compromised hosts, are not clear but there are several possibilities. Previous data have shown that hypoxia, a condition prevalent within large tumors, reduces viral replication (Pipiya et al., “Hypoxia Reduces Adenoviral Replication in Cancer Cells by Downregulation of Viral Protein Expression,” Gene Ther 12:911-917 (2005); Shen et al., “Effect of Hypoxia on Ad5 Infection, Transgene Expression and Replication,” Gene Ther 12:902-910 (2005), which are hereby incorporated by reference in their entirety). Virus spread may also be impaired by the presence of stromal cells and stromal matrix within tumors. Human adenovirus replicate very poorly in murine cells, and murine stromal cells may limit viral spread in xenografts. The aim of this work was to determine if matrix components within tumors might impede the spread of replicating adenovirus.

Previous studies that support this supposition are the findings by Brown et al that collagen can provide a diffusive hindrance in the penetration of therapeutic molecules within tumors, and that this can be improved by matrix modification (Brown et al., “Dynamic Imaging of Collagen and its Modulation in Tumors In Vivo Using Second-harmonic Generation,” Nat Med 9: 796-800 (2003), which is hereby incorporated by reference in its entirety). Also, direct administration of collagenase/dispase or trypsin into glioma xenografts has been shown to enhance the extent of infection of a non-replicating adenoviral vector expressing a reporter gene (Kuriyama et al., “Pretreatment with Protease is a Useful Experimental Strategy for Enhancing Adenovirus-mediated Cancer Gene Therapy,” Hum Gene Ther 11: 2219-2230 (2000), which is hereby incorporated by reference in its entirety).

Cell culture inserts made of polyethylene terephthalate and coated with ECM components have proved to be effective in vitro model systems to study the effects of ECM components in many research areas, including models to study intercellular communication, angiogenesis, metastasis and the inflammatory response. This system was used to study the influence of matrix components on the diffusion of adenovirus in vitro. These experiments demonstrated that collagen I, a major stromal component within tumors, significantly blocked adenoviral diffusion through the insert. In contrast, collagen IV, fibronectin, and laminin did not show any effect on adenoviral diffusion. Strikingly, when collagen I on the insert was treated with collagenase, the ability the adenoviruses to diffuse through the insert was restored. It was confirmed that this blocking effect was not associated with the adenovirus binding to collagen I, but limitation of the passage of virus particles through the membrane by collagen I.

In vitro studies of this kind clearly have limitations, the most major limitation being how well the layer of collagen on a membrane reflects the structure of collagen I within a tumor. This question is difficult to answer, so a non-replicating virus expressing MMP-8 was constructed for further studies. MMP-8 was chosen to break down collagen because MMP-8 has high activity and some selectivity towards collagen I, and has previously been shown to reduce the amount of collagen in several situations. The administration of MMP-8 directed by a non-replicating adenovirus has been shown to reduce the fibrosis in cirrhotic mouse livers (Siller-Lopez et al., “Truncated Active Matrix Metalloproteinase-8 Gene Expression in HepG2 Cells is Active Against Native Type I Collagen,” J Hepatol 33:758-763 (2000); Siller-Lopez et al., “Treatment with Human Metalloproteinase-8 Gene Delivery Ameliorates Experimental Rat Liver Cirrhosis,” Gastroenterology 126:1122-1133; discussion 1949 (2004); Garcia-Banuelos et al, “Cirrhotic Rat Livers with Extensive Fibrosis Can be Safely Transduced with Clinical-grade Adenoviral Vectors. Evidence of Cirrhosis Reversion,” Gene Ther 9:127-134 (2002), which are hereby incorporated by reference in their entirety). MMP-8 activity has also been associated with collagen degradation in humans. In particular secretion of MMP-8 by neutrophils may play a role in resolving fibrotic scar formation during cholestasis (Harty et al., “Repair After Cholestatic Liver Injury Correlates with Neutrophil Infiltration and Matrix Metalloproteinase 8 Activity,” Surgery 138:313-320 (2005), which is hereby incorporated by reference in its entirety), and MMP-8 may play a role in involution of the postpartum uterus (Balbin et al., “Collagenase 2 (MMP-8) Expression in Murine Tissue-remodeling Processes. Analysis of its Potential Role in Postpartum Involution of the Uterus,” J Biol Chem 273:23959-23968 (1998), which is hereby incorporated by reference in its entirety).

A recombinant adenovirus, AdMMP8, that expresses the human MMP-8 gene was therefore constructed and evaluated in an in vitro fibrillar collagen model and a murine human lung cancer A549 cell xenograft model. Fibrillar collagen like amorphous collagen blocked viral diffusion, however, when the fibrillar collagen was conditioned with A549 cells infected by AdMMP8 diffusion was restored. In the A549 cell xenograft model the co-administration of the non-replicating AdMMP8 virus together with a wild type replicating adenovirus resulted in a reduction of collagen visible within necrotic areas of the tumors and significantly reduced tumor growth when compared with wild-type adenovirus infected control tumors. The co-administration of a replicating virus together with non-replicating AdMMP8 is likely to enable replication of the replicating deficient virus (Thorne et al., “Selective Intratumoral Amplification of an Antiangiogenic Vector by an Oncolytic Virus Produces Enhanced Antivascular and Anti-tumor Efficacy,” Mol Ther 13:938-946 (2006), which is hereby incorporated by reference in its entirety).

These finding therefore support the hypothesis that matrix components, in particular collagen I, limit viral diffusion and spread within tumors. The results of the in vitro experiments and the properties of collagen as structural components would suggest that this is a physical property of collagen. In addition, others have shown that collagen I fibers within a tumor have an important effect on diffusive transport within tumors, and can be modulated by collagen dissolution using the hormone relaxin which upregulates several MMPs Brown et al., “Dynamic Imaging of Collagen and its Modulation in Tumors In Vivo Using Second-harmonic Generation,” Nat Med 9:796-800 (2003), which is hereby incorporated by reference in its entirety). Relaxin expression from a replicating adenovirus has also recently been shown to improve tumor responses and viral spread in murine models (Kim et al., “Relaxin Expression from Tumor-targeting Adenoviruses and its Intratumoral Spread, Apoptosis Induction, and Efficacy,” J Natl Cancer Inst 98:1482-1493 (2006), which is hereby incorporated by reference in its entirety).

The data show MMP-8 alone can improve tumor responses in combination with a single low dose of a replicating virus, and that this is associated with reduced amounts of collagen within the tumor. In addition to collagen degradation other mechanisms may also play a role for the positive effect of MMP-8. For instance, cytokines can be bound to matrix components and released by the activities of MMPs. Also MMP-8 has been shown to have anti-inflammatory effects during allergen-induced lung inflammation, partly due to a regulation of inflammatory cell apoptosis (Gueders et al., “Matrix Metalloproteinase-8 Deficiency Promotes Granulocytic Allergen-induced Airway Inflammation,” J Immunol 175:2589-2597 (2005), which is hereby incorporated by reference in its entirety), and may also protect against lethal endotoxin induced hepatitis (Van Lint et al., “Resistance of Collagenase-2 (matrix metalloproteinase-8)-Deficient Mice to TNF-Induced Lethal Hepatitis,”J Immunol 175:7642-7649 (2005), which is hereby incorporated by reference in its entirety). Although clear changes in collagen composition within the tumors were noticed, no obvious changes in inflammatory cell infiltrates within these tumors in nude mice were evident.

The actions of some of the matrix metalloproteinases have been found to have an important role in tumor invasion and metastases. This is a potential deleterious effect of the administration of MMP-8. However, so far MMP-8 has not been associated in a positive way with tumorigenesis or metastasis. In fact, in isogenic breast cancer cell lines expression of MMP-8 has been associated with inhibition of metastases and invasion (Agarwal et al., “Expression of Matrix Metalloproteinase 8 (MMP-8) and Tyrosinase-related Protein-1 (TYRP-1) Correlates with the Absence of Metastasis in an Isogenic Human Breast Cancer Model,” Differentiation 71:114-125 (2003), which is hereby incorporated by reference in its entirety). Further, genetic manipulation of a metastatic cell line to up-regulate the activity of MMP-8 gene has been shown to decrease metastatic spread, and conversely, its down-regulation by incorporation of a targeted ribozyme into a nonmetastatic line resulted in the cells becoming metastatic (Montel et al., “Altered Metastatic Behavior of Human Breast Cancer Cells After Experimental Manipulation of Matrix Metalloproteinase 8 Gene Expression,” Cancer Res 64:1687-1694 (2004), which is hereby incorporated by reference in its entirety). Mice deficient in MMP-8 (MMP-8^{-/-} mice) have been reported to have an increased incidence of skin tumors (Balbin et al., “Loss of Collagenase-2 Confers Increased Skin Tumor Susceptibility to Male Mice,” Nat Genet 35:252-257 (2003), which is hereby incorporated by reference in its entirety), and female MMP-8^{-/-} mice submitted to oopherectomy or treated with tamoxifen were more susceptible to tumors compared with wild-type mice. Further, in this study, bone marrow transplantation experiments revealed that MMP-8 supplied by neutrophils was sufficient to restore the natural protection against tumor development mediated by this MMP-8 in this model (Balbin et al., “Loss of Collagenase-2 Confers Increased Skin Tumor Susceptibility to Male Mice,” Nat Genet 35:252-257 (2003), which is hereby incorporated by reference in its entirety).

The data also suggest that MMP-8 expression has no deleterious effect on cancer progression. AdMMP8 treated tumors in the absence of wild type virus grew with similar growth kinetics to the control group. There was also no evidence of metastatic spread to the lung of A549 tumors after treatment of mice with Adwt300/AdMMP-8. All these findings suggest that there was no increase in tumor invasiveness or enhanced tumor cell spread after AdMM-8 administration, and a recent report has also shown a decreased metastatic potential of tumors treated with a replicating virus expressing the hormone relaxin that activate MMPs (Kim et al., “Relaxin Expression from Tumor-targeting Adenoviruses and its Intratumoral Spread, Apoptosis Induction, and Efficacy,” J Natl Cancer Inst 98:1482-1493 (2006), which is hereby incorporated by reference in its entirety).

In conclusion, incorporation of the MMP-8 transgene within a replicating adenovirus might be a beneficial strategy for improving viral spread and improving oncolytic activity of a replicating adenovirus.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of treating cancer in a subject, said method comprising:
co-administering a replicating virus and a matrix metalloproteinase to the subject under conditions effective to treat cancer.

2. The method according to claim 1, wherein the matrix metalloproteinase is one or more of metalloproteinase-1 to 28.

3. The method according to claim 2, wherein the matrix metalloproteinase is human matrix metalloproteinase-8.

4. The method according to claim 1, wherein the matrix metalloproteinase is administered as a nucleic acid encoding the matrix metalloproteinase which is operatively positioned in the replicating virus under conditions effective to express the matrix metalloproteinase.

5. The method according to claim 1, wherein the replicating virus is selected from the group consisting of a replicating adenovirus, reovirus, a replicating herpesvirus, vaccinia, measles, and vesicular stomatitis.

6. The method according to claim 5, wherein the replicating virus is a replicating adenovirus.

7. The method according to claim 1, wherein the cancer is selected from the group consisting of lung cancer, colon cancer, breast cancer, prostate cancer, pancreatic cancer, and glioma cancer.

8. The method according to claim 1, wherein the subject is a human.

9. The method according to claim 1, wherein the matrix metalloproteinase is administered as a nucleic acid encoding the matrix metalloproteinase, said nucleic acid is operatively positioned in a non-replicating virus.

10. The method according to claim 9, wherein the non-replicating virus is selected from the group consisting of adeno-associated virus, a non-replicating adenovirus, and a non-replicating herpesvirus.

11. The method according to claim 1, wherein the matrix metalloproteinase is administered as a protein.

12. A method of enhancing the delivery to and distribution within a tumor mass of therapeutic viruses, said method comprising:
co-administering a replicating virus and a matrix metalloproteinase to the tumor mass under conditions effective to enhance the delivery to and distribution within the tumor mass of therapeutic viruses.

13. The method according to claim 12, wherein the matrix metalloproteinase is one or more of metalloproteinase-1 to 28.

14. The method according to claim 13, wherein the matrix metalloproteinase is human matrix metalloproteinase-8.

15. The method according to claim 12, wherein the matrix metalloproteinase is administered as a nucleic acid encoding the matrix metalloproteinase which is operatively positioned in the replicating virus under conditions effective to express the matrix metalloproteinase.

16. The method according to claim 12, wherein the replicating virus is selected from the group consisting of a replicating adenovirus, reovirus, a replicating herpesvirus, vaccinia, measles, and vesicular stomatitis.

17. The method according to claim 16, wherein the replicating virus is a replicating adenovirus.

18. The method according to claim 12, wherein the tumor is associated with a cancer selected from the group consisting of lung cancer, colon cancer, breast cancer, prostate cancer, pancreatic cancer, and glioma cancer.

19. The method according to claim 12, wherein the replicating virus and the matrix metalloproteinase are administered to a subject.

20. The method according to claim 19, wherein the subject is a human.

21. The method according to claim 12, wherein said co-administering prevents disruption of delivery to and distribution within a tumor mass by collagen I.

22. The method according to claim 12, wherein the matrix metalloproteinase is administered as a nucleic acid encoding the matrix metalloproteinase, said nucleic acid is operatively positioned in a non-replicating virus.

23. The method according to claim 22, wherein the non-replicating virus is selected from the group consisting of adeno-associated virus, a non-replicating adenovirus, and a non-replicating herpesvirus.

24. The method according to claim 12, wherein the matrix metalloproteinase is administered as a protein.

25. A cancer therapeutic comprising:
a replicating virus and
a matrix metalloproteinase.

26. The cancer therapeutic according to claim 25, wherein the matrix metalloproteinase is one or more of metalloproteinase-1 to 28.

27. The cancer therapeutic according to claim 26, wherein the matrix metalloproteinase is human matrix metalloproteinase-8.

28. The cancer therapeutic according to claim 25, wherein the replicating virus is selected from the group consisting of a replicating adenovirus, reovirus, a replicating herpesvirus vaccinia, measles, and vesicular stomatitis.

29. The cancer therapeutic according to claim 28, wherein the replicating virus is a replicating adenovirus.

30. The cancer therapeutic according to claim 25 further comprising:
a pharmaceutically acceptable carrier and/or adjuvant.

31. The cancer therapeutic according to claim 25, wherein the matrix metalloproteinase is in the form of a nucleic acid molecule encoding the matrix metalloproteinase, said nucleic acid being operatively positioned in the replicating virus under conditions effective to express the matrix metalloproteinase.

32. The cancer therapeutic according to claim 25, wherein the matrix metalloproteinase is in the form of a nucleic acid molecule encoding the matrix metalloproteinase, said nucleic acid being operatively positioned in a non-replicating virus under conditions effective to express the matrix metalloproteinase.

33. The cancer therapeutic according to claim 32, wherein the non-replicating virus is selected from the group consisting of adeno-associated virus, a non-replicating adenovirus, and a non-replicating herpesvirus.

34. The cancer therapeutic according to claim 25, wherein the matrix metalloproteinase is in the form of a protein mixed with the replicating virus.
